(19) United States
(12) Patent Application Publication
(10) Pub. No.: US 2016/0108421 A1
(43) Pub. Date: Apr. 21, 2016

(54) METHOD FOR PRODUCING STRESS TOLERANT TRANSGENIC PLANT BY SILENCING A GENE ENCODING CALCIUM-DEPENDENT LIPID-BINDING PROTEIN WITH C2 DOMAIN AND APPLICATIONS OF THE SAME

(71) Applicant: Board of Trustees of the University of Arkansas, Little Rock, AR (US)

(72) Inventors: Mariya Khodakovskaya, Little Rock, AR (US); Virginia Ann Boyett-Johnson, Perryville, AR (US)

(21) Appl. No.: 14/977,164

(22) Filed: Dec. 21, 2015

Related U.S. Application Data
(63) Continuation-in-part of application No. 14/030,404, filed on Sep. 18, 2013.
(60) Provisional application No. 61/703,597, filed on Sep. 20, 2012.

(51) Int. Cl. C12N 15/82

(52) U.S. Cl. CPC C12N 15/8273; C12N 15/8271

(57) ABSTRACT

A method for silencing rice plant, includes the steps of: identifying a first nucleotide sequence from Oryza sativa, wherein the first nucleotide sequence is homologous to a Ca²⁺-dependent lipid-binding gene of Arabidopsis thaliana (Atclb gene); cloning the first nucleotide sequence to a first vector to form a first recombinant vector, such that the first recombinant vector contains a first insert of the first nucleotide sequence; and transfer the first insert in the first recombinant vector to a second vector to form a second recombinant vector, such that the second recombinant vector contains a second insert of the first nucleotide sequence. The second recombinant vector, when being introduced to Oryza sativa plant cells, is capable of silencing the first nucleotide sequence of the Oryza sativa plant cells, such that the Oryza sativa plant cells are tolerant to an abiotic stress.

### CROSS-REFERENCE TO RELATED APPLICATIONS

This application is a continuation-in-part application of the U.S. application Ser. No. 14/030,404, filed Sep. 18, 2013, which itself claims priority to and the benefit of, pursuant to 35 U.S.C. §119(e), U.S. Provisional Application Ser. No. 61/703,597, filed on Sep. 20, 2012, which are incorporated herein in their entireties by reference.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### FIELD OF THE INVENTION

The present invention relates generally to a method for producing a transgenic plant and application of same, and more particularly to a method for increasing stress tolerance of a plant by silencing a gene encoding calcium-dependent lipid-binding protein with C2 domain and a plant produced thereof.

### BACKGROUND OF THE INVENTION

All abiotic stresses (cold, drought, salt) reduce plant growth and yield. These are the major limiting factors in agricultural productivity and quality, thus preventing crop plants from realizing their full genetic potential.

Transgenic or recombinant plants are of increasing interest because of the potential to control phenotypic traits as well as to produce large quantities of commercially useful products. Plants have been employed to overproduce heterologous proteins and in principle can produce a wide range of products, including high value proteins and certain pharmaceuticals. Transgenic plants with enhanced production traits such as the ability to survive and thrive after exposure to prolonged environmental stress are particularly desirable as a source of economic benefit to the commercial agricultural plant community including horticulturists, food producers, and agronomist. The identification of new genes that can effectively control stress response in plants is one of the major goals of plant biotechnology. Unfortunately, in many cases overexpression of genes involved in stress signaling can lead to morphological abnormalities, restricting the potential for use in commercial production.

Therefore, a heretofore unaddressed need exists in the art to address the aforementioned deficiencies and inadequacies.

### SUMMARY OF THE INVENTION

In one aspect, the present invention is directed to a transgenic plant.

In one embodiment, the transgenic plant includes a first gene having a first polynucleotide and a recombinant construct having a second polynucleotide. The first polynucleotide is homologous to a Atclb gene fragment represented by nucleotides 963-1205 of SEQ ID NO:1. The second polynucleotide has at least 90% identity to the first polynucleotide or a sequence complementary to the first polynucleotide. The recombinant construct is configured to silence the first gene such that the transgenic plant is tolerant to abiotic stress.

In one embodiment, the first polynucleotide has at least 50% similarity to the Atclb gene fragment.

In one embodiment, the first polynucleotide encodes a C2-like domain, and the C2-like domain has at least 50% amino acid identity to amino acids 265-345 of SEQ ID NO: 2.

In one embodiment, the second polynucleotide is substantially identical to the first polynucleotide or the sequence complementary to the first polynucleotide.

In one embodiment, the recombinant construct is configured to silence the first gene of the transgenic plant by RNA interference, anti-sense construction or virus induced gene silencing (VIGS).

In one embodiment, the abiotic stress is water deficit, salt stress, cold stress or a combination thereof.

In one embodiment, the transgenic plant is formed from a plant of Solanum lycopersicum, Ricinus communis, Populus trichocarpa, Vitis vinifera, Oryza sativa, or Sorghum bicolor, and the identified genes are SlCLB1, RcCLBP, PtIMSCDP, VvHP, OsHP, or SbIMSCDP, respectively.

In one embodiment, the transgenic plant is formed from the Oryza sativa, and the first gene is OsHP gene encoding Oryza sativa histidine-containing phosphotransfer protein.

In one embodiment, the transgenic plant is formed from the Solanum lycopersicum, and the first gene is SlCLB1 gene encoding a protein with a Ca²⁺-dependent lipid-binding domain.

In one embodiment, a seed is produced by the transgenic plant.

In one embodiment, the present invention is directed to a transgenic plant. The transgenic plant includes a mutated first gene such that the transgenic plant is tolerant to abiotic stress. The first gene to be mutated has a first polynucleotide. The first polynucleotide is homologous to a Atclb gene fragment represented by nucleotides 963-1205 of SEQ ID NO:1.

In one aspect, the present invention is directed to a method of producing transformed plant cells.

In one embodiment, the method includes: identifying a first gene of plant cells; constructing a recombinant construct for silencing the first gene in the plant cells; and transforming the plant cells with the recombinant construct to form the transformed plant cells. The first gene in the transformed plant cell is silenced by the recombinant construct such that the plant cells are tolerant to an abiotic stress. The first gene includes a first polynucleotide, and the first polynucleotide is homologous to a Atclb gene fragment represented by nucleotides 963-1205 of SEQ ID NO:1. The recombinant construct includes a second polynucleotide, and the second polynucleotide has at least 90% similarity to the first polynucleotide or a sequence complementary to the first polynucleotide.

In one embodiment, the method further includes confirming the silencing of the first gene in the transformed plant cells.

In one embodiment, the method further includes producing a plant from the transformed plant cells.

In one embodiment, the first polynucleotide has at least 50% similarity to the Atclb gene fragment.

In one embodiment, the first polynucleotide encodes a C2-like domain, and the C2-like domain has at least 50% amino acid identity to amino acids 265-345 of SEQ ID NO: 2.

In one embodiment, the second polynucleotide is substantially identical to the first polynucleotide or the sequence complementary to the first polynucleotide.

In one embodiment, the plant cells are Solanum lycopersicum cells, Ricinus communis cells, Populus trichocarpa cells, Vitis vinifera cells, Oryza sativa cells, or Sorghum bicolor cells.

In one embodiment, the plant cells are the Oryza sativa cells, and the first gene is OsHP gene encoding Oryza sativa histidine-containing phosphotransfer protein.

In one embodiment, the plant cells are the Solanum lycopersicum cells, and the first gene is SlCLB1 gene encoding a protein with a Ca²⁺-dependent lipid-binding domain.

In one embodiment, the recombinant construct is configured to silence the first gene of the plant cells by RNA interference, anti-sense construction or virus induced gene silencing (VIGS).

In one embodiment, the abiotic stress is water deficit, salt stress, cold stress or a combination thereof.

In one embodiment, the plant cells are transformed by agrobacterial transformation or bombardment transformation to form the transformed plant cells.

In one embodiment, the present invention is directed to a transgenic plant produced from the transformed plant cells.

In one embodiment, the present invention is directed to a seed produced by the transgenic plant.

In one aspect, the present invention is directed to a method of producing transformed plant cells.

In one embodiment, the method includes mutating a first gene of plant cells to form the transformed plant cells such that the transformed plant cells are tolerant to an abiotic stress. A first polynucleotide of the first gene is homologous to Atclb gene fragment represented by nucleotides 963-1205 of SEQ ID NO:1.

In one aspect, the present invention relates to a method for silencing rice plant. The method includes:

identifying a first nucleotide sequence from Oryza sativa, wherein the first nucleotide sequence is homologous to a Ca²⁺-dependent lipid-binding gene of Arabidopsis thaliana (Atclb gene);

cloning the first nucleotide sequence to a first vector to form a first recombinant vector, such that the first recombinant vector contains a first insert of the first nucleotide sequence; and

transfer the first insert in the first recombinant vector to a second vector to form a second recombinant vector, such that the second recombinant vector contains a second insert of the first nucleotide sequence,

where the second recombinant vector, when being introduced to Oryza sativa plant cells, is capable of silencing the first nucleotide sequence of the Oryza sativa plant cells, such that the Oryza sativa plant cells are tolerant to an abiotic stress.

In one embodiment, the first nucleotide sequence is homologous to nucleotide sequence 963-1205 of SEQ ID NO:1.

In one embodiment, the first nucleotide sequence encodes Oryza sativa histidine-containing phosphotransfer protein, and has the sequence of SEQ ID NO: 10.

In one embodiment, the first vector is an entry vector pENTR™/D-TOPO, and the step of cloning the first nucleotide sequence to the first vector is a BP reaction.

In one embodiment, the first insert has the sequence of SEQ ID NO:11. In other embodiments, the first insert may also be nucleotide sequence that is complementary to SEQ ID NO:11.

In one embodiment, the method further includes transforming E. coli competent cells by the first recombinant vector to form transformed E. coli competent cells, where the transformed E. coli competent cells were incubated at room temperature.

In one embodiment, the method further includes transforming E. coli competent cells by the second recombinant vector to form transformed E. coli competent cells, where the transformed E. coli competent cells were incubated at room temperature.

In one embodiment, the second vector is a pANDA destination vector, and the step of transfer the first insert in the first recombinant vector to the second vector is an LR reaction.

In one embodiment, the method further includes transforming the Oryza sativa plant cells using the second recombinant vector to form transformed Oryza sativa plant cells.

In one embodiment, the Oryza sativa plant cells are transformed by agrobacterial transformation or bombardment transformation to form the transformed Oryza sativa plant cells.

In one embodiment, the method further includes, before the step of cloning the first nucleotide sequence to the first vector to form the first recombinant vector, amplifying the first nucleotide sequence using a pair of primers that have the nucleotide sequences of SEQ ID NO:12 and SEQ ID NO:13.

In one embodiment, the abiotic stress is water deficit, salt stress, cold stress or a combination thereof.

These and other aspects of the present invention will become apparent from the following description of the preferred embodiment taken in conjunction with the following drawings, although variations and modifications therein may be effected without departing from the spirit and scope of the novel concepts of the disclosure.

### BRIEF DESCRIPTION OF THE DRAWINGS

The accompanying drawings illustrate one or more embodiments of the invention and, together with the written description, serve to explain the principles of the invention. Wherever possible, the same reference numbers are used throughout the drawings to refer to the same or like elements of an embodiment.

FIG. 1 illustrates a method for producing a plant tolerant to stress conditions by silencing a gene in the plant that is homologous to Arabidopsis thaliana Atclb gene, according to one embodiment of the present invention.

FIG. 2A illustrate the schematic structure of the AtCLB protein, according to one embodiment of the present invention.

FIG. 2B illustrate the amino acid sequence alignment of C2 domain of the AtCLB protein with its homologues from different plant species, according to one embodiment of the present invention.

FIG. 2C illustrate phylogenetic analysis of the C2 domains from the different plant species, according to one embodiment of the present invention.

FIGS. 3A-3E illustrate drought-tolerance phenotype of T-DNA insertion knockout lines Atclb1-1 and Atclb1-2 produced according to one embodiment of the present invention.

FIG. 3A is a schematic diagram of AtCLB genomic structure and T-DNA insertion site in the mutant allele Atclb1, according to one embodiment of the present invention.

FIG. 3B illustrate germination rate of Arabidopsis thaliana wild type (WT), a line expressing Atclb under the control of the 35S promoter, and two knockout mutant lines after 1 week of incubation on Murashige and Skoog medium (MS medium), according to one embodiment of the present invention.

FIG. 3C illustrate phenotype of wild type (WT) and knockout mutant lines after 3 weeks of withholding water, according to one embodiment of the present invention.

FIG. 3D illustrate relative leaf water content of 4-week-old wild type (WT) and two knockout mutant lines during 3 weeks of water deficit stress, according to one embodiment of the present invention.

FIG. 3E illustrate expression analysis of wild type and two homozygous knockout lines (Atclb1-1 and Atclb1-2 of thaliana) by RT-PCR, according to one embodiment of the present invention.

FIGS. 4A-4C illustrate salt-tolerance phenotype of T-DNA insertion knockout lines Atclb1-1 and Atclb1-2 produced according to one embodiment of the present invention.

FIG. 4A illustrate germination of Arabidopsis thaliana wild type (WT) and Atclb1-1 line in the presence of 150 millimolar (mM) sodium chloride (NaCl), according to one embodiment of the present invention.

FIG. 4B illustrate seedings of loss-of-function Atclb1-1 mutant line were able to survive in the presence of 150 mM NaCl, according to one embodiment of the present invention.

FIG. 4C illustrate one-week-old Arabidopsis thaliana seedlings with 10 to 20 millimeter (mm) long roots on vertical agar plates were transferred to plates supplemented with NaCl and allowed to grow vertically at 23° C. with a 16-h light period, according to one embodiment of the present invention.

FIG. 5 illustrates a method for producing a rice plant tolerant to stress conditions by silencing a gene from rice plant that is homologous to Arabidopsis thaliana Atclb gene, according to one embodiment of the present invention.

FIG. 6 illustrates a method for producing a rice plant tolerant to stress conditions by silencing a gene from rice plant that is homologous to Arabidopsis thaliana Atclb gene, according to one embodiment of the present invention.

FIG. 7 illustrates a method for producing a tomato plant tolerant to stress conditions by silencing a gene from tomato plant that is homologous to Arabidopsis thaliana Atclb gene, according to one embodiment of the present invention.

### DETAILED DESCRIPTION OF THE INVENTION

The present invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this invention will be thorough and complete, and will fully convey the scope of the invention to those skilled in the art. Like reference numerals refer to like elements throughout.

As used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which has no influence on the scope of the invention. Additionally, some terms used in this specification are more specifically defined below.

As used herein, the terms “comprise or comprising”, “include or including”, “have or having”, “contain or containing” and the like are to be understood to be open-ended, i.e., to mean including but not limited to.

Typically, terms such as “first”, “second”, “third”, and the like are used for distinguishing various elements, members, regions, layers, and areas from others. Therefore, the terms such as “first”, “second”, “third”, and the like do not limit the number of the elements, members, regions, layers, areas, or the like. Further, for example, the term “first” can be replaced with the term “second”, “third”, or the like.

Typically, terms such as “about,” “approximately,” “generally,” “substantially,” and the like unless otherwise indicated mean within 20 percent, preferably within 10 percent, preferably within 5 percent, and even more preferably within 3 percent of a given value or range. Numerical quantities given herein are approximate, meaning that the term “about,” “approximately,” “generally,” or “substantially” can be inferred if not expressly stated.

As used herein, “AtCLB” refers to a Ca²⁺-dependent lipid-binding protein from Arabidopsis thaliana encoded by Atclb gene of Arabidopsis thaliana. AtCLB contains a Ca²⁺-binding domain or C2 domain. AtCLB may be a transcriptional regulator in the Ca²⁺ signaling pathway.

As used herein, an “Atclb gene” from Arabidopsis thaliana encodes the protein AtCLB. Atclb gene is identified as a novel and negative regulator for stress tolerance that can negatively regulate response to abiotic stress in Arabidopsis thaliana.

A protein “homologous” to AtCLB protein may have at least 30% identical or conserved amino acid sequence in comparison to AtCLB protein sequence. Preferably, the sequence identity or similarity between the homologous protein sequence and AtCLB protein sequence is more than 60%. More preferably, the sequence identity or similarity between the homologous protein sequence and AtCLB protein sequence is more than 90%. Further, the sequence identity or similarity between the homologous protein sequence and AtCLB protein sequence is more than 95%.

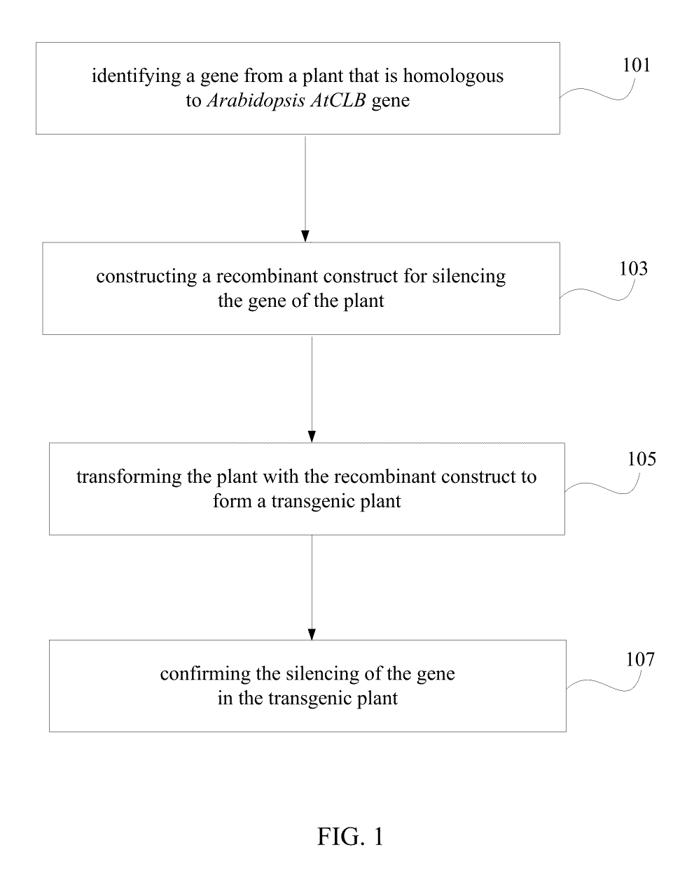

The description will be made as to the embodiments of the present invention in conjunction with the accompanying drawings in FIGS. 1-6. In accordance with the purposes of this invention, as embodied and broadly described herein, this invention, in one aspect, relates to method for producing transgenic plant.

In one aspect the present invention, a method for increasing plant productivity by enhancement of tolerance to stressful environmental conditions, especially abiotic stress including water deficit stress, cold and salt stress is provided. The method includes applying genetic approach and silencing key genes involved in plant stress signaling.

Mutation in Arabidopsis thaliana Atclb gene encoding calcium-dependent protein with a C2 domain (AtCLB) leads to significant increase of abiotic stress tolerance in Arabidopsis thaliana plant. Thus, lack of expression of Atclb gene with following lack of production of AtCLB protein leads to increase of tolerance to water deficit and salt stresses in Atclb mutants of Arabidopsis thaliana.

Many valuable crops have homologues of Atclb gene. Bioinformatics analysis revealed genes from important crops including tomato and rice, which include sequences identical/similar to Atclb gene fragment encoding C2 domain. Increasing crop tolerance to stressful environmental conditions in valuable crops may be achieved by silencing of those genes homologues to Atclb gene.

In one embodiment, the plant is Solanum lycopersicum, and the gene homologous to Atclb gene is SlCLB1, the plant is Ricinus communis, and the gene homologous to Atclb gene is RcCLBP, the plant is Populus trichocarpa, and the gene homologous to Atclb gene is PtIMSCDP, the plant is Vitis vinifera, and the gene homologous to Atclb gene is VvHP, the plant is Oryza sativa, and the gene homologous to Atclb gene is OsHP, or the plant is Sorghum bicolor, and the gene homologous to Atclb gene is SbIMSCDP.

In one embodiment, rice has the OsHP gene encoding Oryza sativa histidine-containing phosphotransfer protein. The OsHP gene is homologous to Atclb gene. Transformation of rice with an established vector construction (for example, Panda-OsHP) may silence rice OsHP gene and increase stress tolerance of a transformed rice plant.

Genetically modified plants carrying silenced homolog of Atclb gene can be used as alternative to traditional crops in areas with stressful environmental conditions (areas with water deficit, areas with salt soil). The production of those transgenic crops, among other things, will enhance productivity of lands and benefit agriculture.

FIG. 1 illustrates a method for producing a plant tolerant to stress conditions by silencing a gene in the plant that is homologous to Arabidopsis thaliana Atclb gene, according to one embodiment of the present invention. The stress may be abiotic stress and includes water deficit, salt stress, cold stress or a combination thereof. The method includes the following steps.

In step 101, a gene homologues to Atclb gene is identified from a plant or crop of choice. In one embodiment, the gene from the plant or crop of choice is identified by bioinformatics analysis. In certain embodiments, the gene has a first polypeptide encoding a C2-like domain. The first polypeptide of the gene is homologous to a fragment of the Atclb gene that encoding C2 domain of the ATCLB protein. The nucleotide fragment encoding C2 domain of ATCLB protein is represented by nucleotides 963-1205 of SEQ ID NO:1, and the C2 domain of ATCLB protein is represented by amino acids 265-345 of SEQ ID NO:2. The first polynucleotide is homologous to the nucleotides 963-1205 of SEQ ID NO:1, and the C2-like domain encoded by the first polynucleotide is homologous to the C2 domain, i.e., amino acids 265-345 of SEQ ID NO:2.

In certain embodiments, the first polynucleotide from the plant of choice has at least about 30% sequence similarity to the Atclb gene fragment encoding C2 domain. Preferably, the sequence similarity is above 50%. More preferably, the sequence similarity/identity is above 90%.

In certain embodiments, the first polynucleotide from the plant of choice has at least about 30% sequence identity to the Atclb gene fragment encoding C2 domain. Preferably, the sequence identity is above 50%. More preferably, the sequence identity is above 90%. In certain embodiments, due to the gene degeneracy, the protein pairwise identity percentage is higher than the corresponding nucleic acid pairwise identity percentage.

In certain embodiments, the C2-like domain from the plant of choice has at least about 30% sequence similarity to the C2 domain of AtCLB protein. Preferably, the sequence similarity is above 50%. More preferably, the sequence similarity/identity is above 90%.

In certain embodiments, the C2-like domain from the plant of choice has at least about 30% sequence identity to the C2 domain of AtCLB protein. Preferably, the sequence identity is above 50%. More preferably, the sequence identity is above 90%. In certain embodiments, due to the gene degeneracy, the protein pairwise identity percentage is higher than the corresponding nucleic acid pairwise identity percentage.

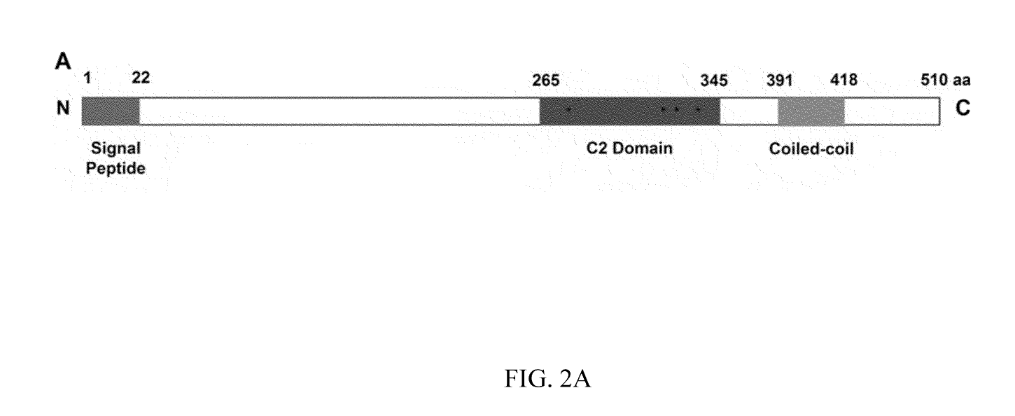

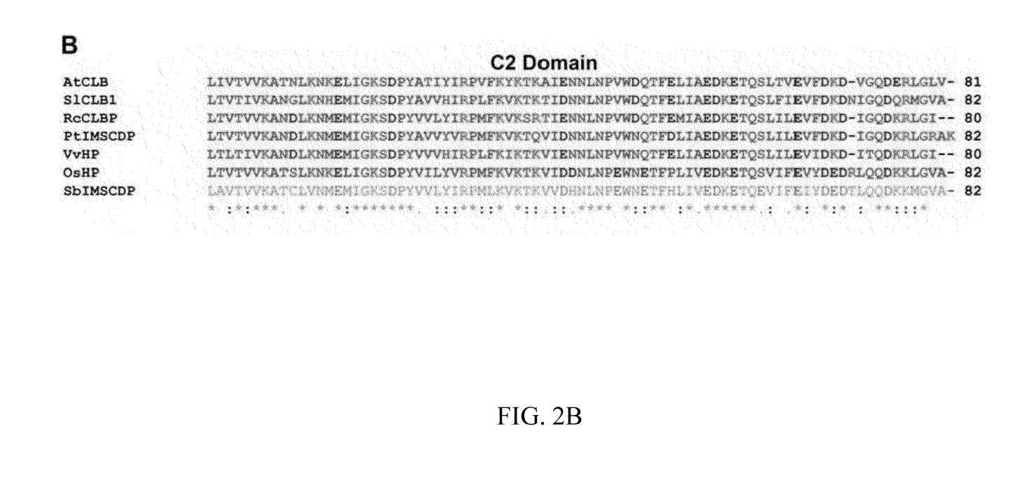

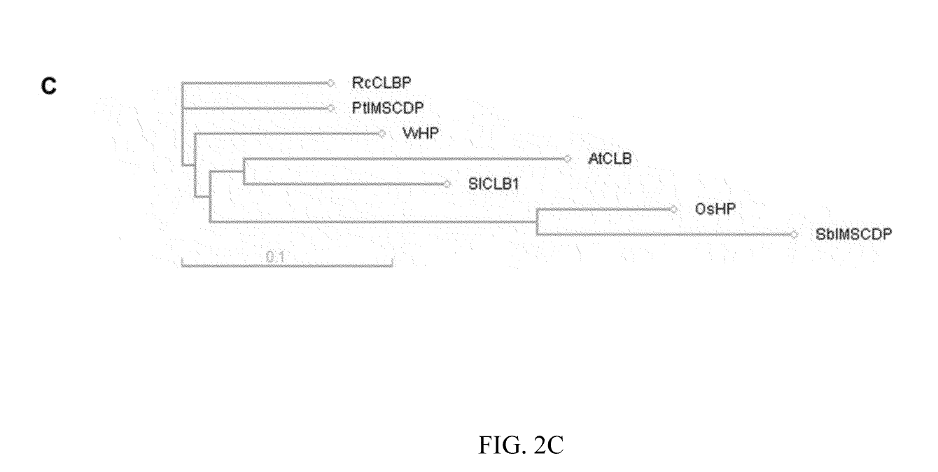

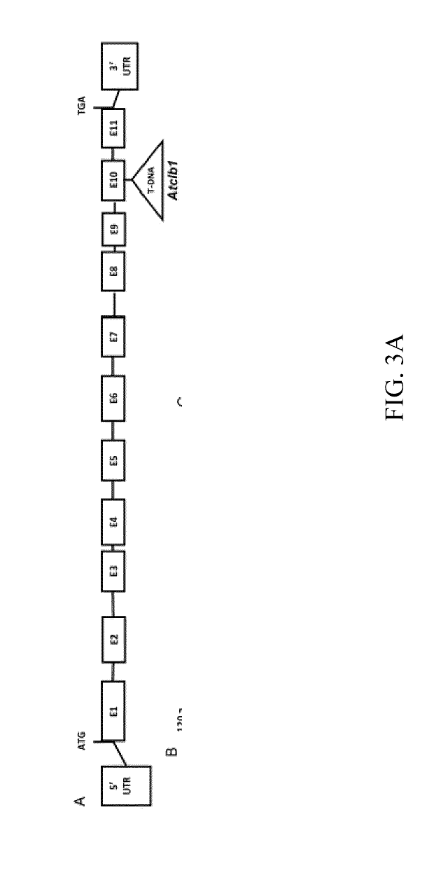

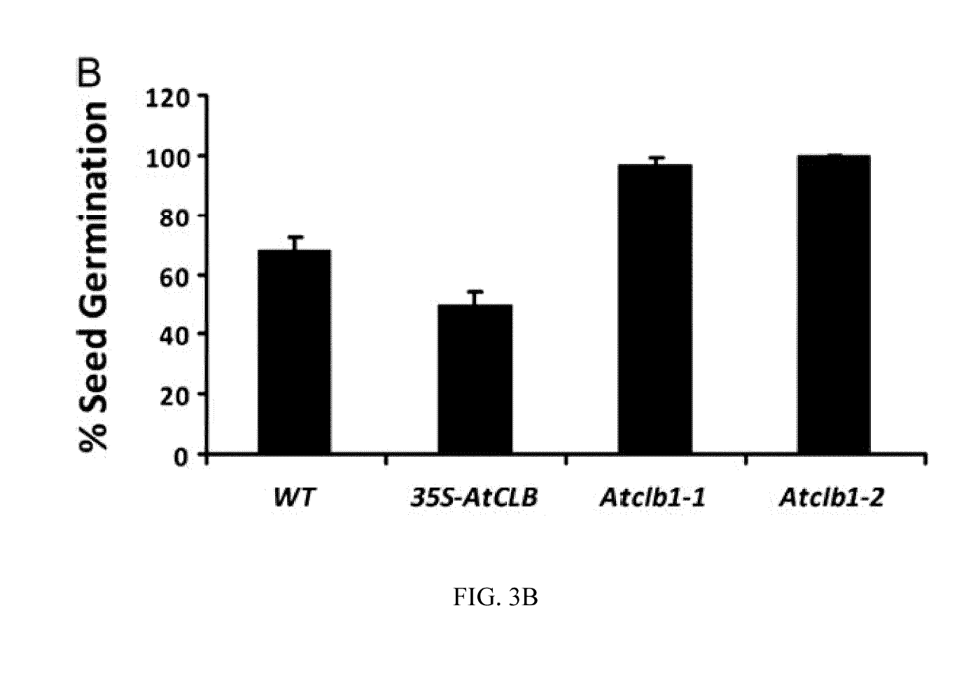

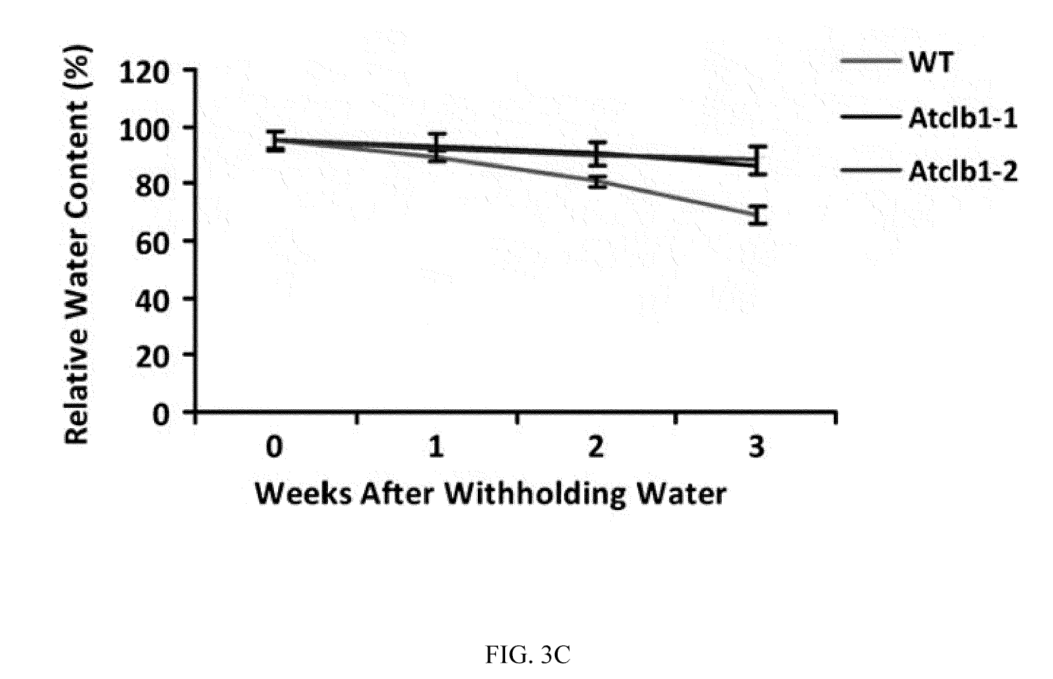

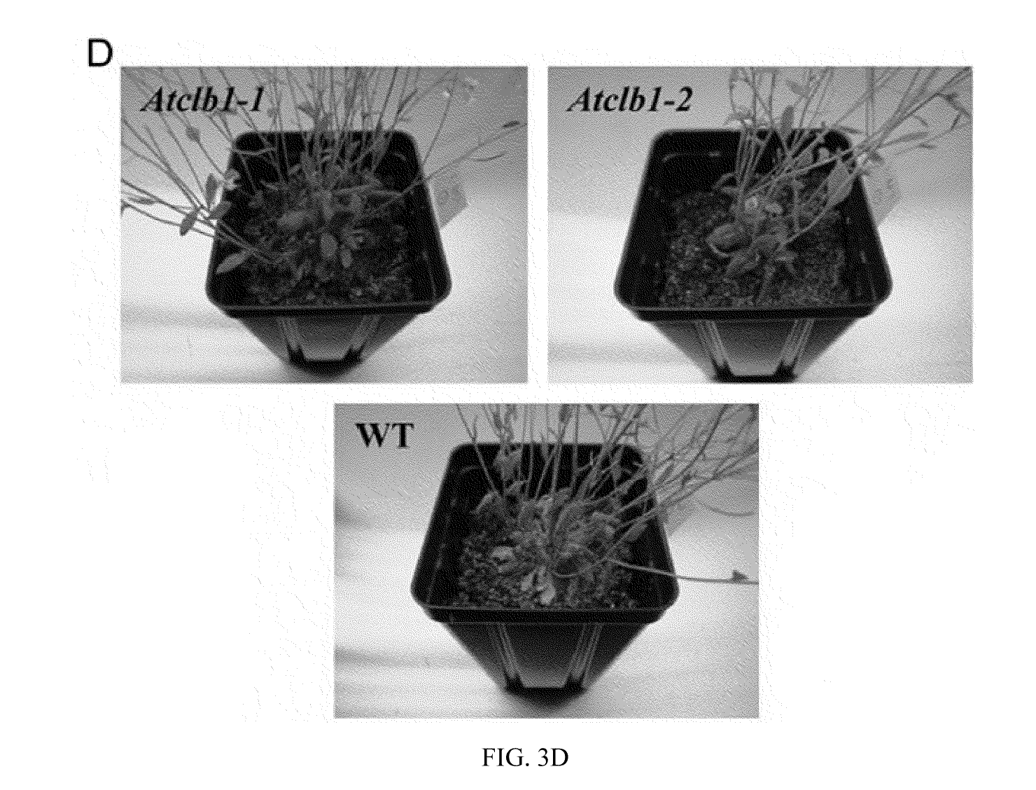

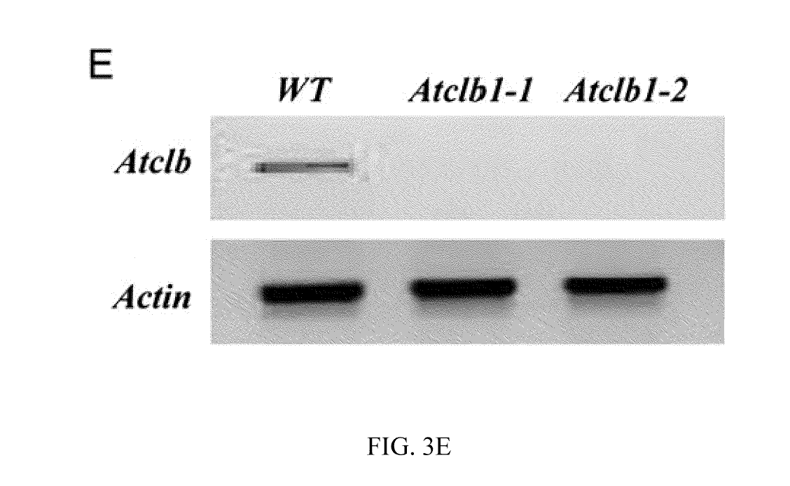

Bioinformatics analysis has revealed that the C2 domain of Arabidopsis thaliana calcium-dependent, lipid-binding protein is homologous to the C2-like domain sequences of important crops, including tomato and rice. Expression analysis of Atclb rice homologue (OsHP) confirmed the presence of the gene transcripts in root, stem, leaf, flower of these crops (data generated but not shown here). T-DNA knockout mutant analysis revealed that down regulation of AtCLB containing a conserved C2 domain could confer resistance to various abiotic stress conditions such as salinity and drought in Arabidopsis thaliana (FIGS. 2 and 3). Silencing of the expression of AtCLB C2 domain homologues in crops proves involvement of conserved C2 domain in stress signaling and potentially brings resistance to abiotic stress conditions.

AtCLB gene was identified using a 240-bp DNA sequence as bait in a yeast one-hybrid screen assay against the cDNA library from Arabidopsis thaliana root apices. The 240-bp DNA sequence is a fragment of the promoter region of AtTHAS1 (Thalianol synthase). The identified AtCLB gene codes for an Arabidopsis thaliana calcium-dependent lipid-binding protein (AtCLB) with a C2 domain. Referring to FIG. 2A, AtCLB is a putative protein containing 510 amino acid residues with a predicted molecular mass of 55 kDa, represented by SEQ ID NO2. The AtCLB protein has a predicted N-terminal signal peptide at positions 1-22, a predicted C-terminal coiled-coil region, and the C2 domain at positions 265-345. The asterisks in the C2 domain represents four of the five conserved aspartic acid residues at positions 285, 333, 335, and 339 that are thought to be involved in Ca²⁺ binding.

Referring to FIG. 2B, identical residues in all sequences in the alignment are denoted by ‘*’ while conserved substitutions and semi-conserved substitutions are denoted by ‘:’ and ‘.’ respectively. The Basic Local Alignment Search Tool (BLAST)® analysis revealed that the C2 domain of AtCLB protein (SEQ ID NO:3) has significant homology to C2-like domain sequences from plant species SlCLB1 gene of plant Solanum lycopersicum, RcCLBP gene of plant Ricinus communis, PtIMSCDP gene of plant Populus trichocarpa, VvHP gene of plant Vitis vinifera, OsHP gene of plant Oryza sativa, and SbIMSCDP gene of plant Sorghum bicolor. The pairwise identities of AtCLB protein with its homologous C2-like domain sequences SlCLB1 (SEQ ID NO:4), RcCLBP (SEQ ID NO:5), PtIMSCDP (SEQ ID NO:6), VvHP (SEQ ID NO:7), OsHP (SEQ ID NO:8), and SbIMSCDP proteins (SEQ ID NO:9) are >57%.

Referring to FIG. 2C, to evaluate the divergence of the C2 domain of AtCLB from other C2-like domains aligned in FIG. 2B, a phylogenetic tree was constructed using C2 domain or C2-like domain sequences of these proteins and it was found that they fall into distinct classes, with AtCLB sharing the same group as SlCLB1. The length of lines connecting the different C2 domain or C2-like domains is a measure of their sequence distance.

In step 103, a recombinant construct, such as a vector, is constructed. The recombinant construct comprises a second polynucleotide. In certain embodiment, the second polynucleotide has at least 90% sequence identity to the first polynucleotide or a sequence complementary to the first polynucleotide. In one embodiment, the second polynucleotide is identical to the first polynucleotide. In one embodiment, the second polynucleotide is identical to the complementary sequence of the first polynucleotide. The recombinant construct is capable of switching off (silencing) the gene in the plant that is homologous to Atclb gene. In one embodiment, the recombinant construct is constructed for RNA silencing, antisense silencing or virus induced gene silencing (VIGS).

In step 105, the plant is transformed with the established recombinant construct by any available transformation technique. In certain embodiments, the transformation method may include agrobacterial transformation and bombardment method.

In certain embodiments, the transformation of the plant has a transient silencing effect. In certain preferred embodiments, the transformation of the plant has a stable silencing effect that can be passed on.

The method may further include a confirmation step to confirm or select transgenic plants with the gene homologous to Atclb gene successfully silenced. In one embodiment, homozygous lines of transgenic plants are tested in abiotic stress experiments and stress tolerant lines can be selected for range of field experiments.

### Example 1

### Tolerance to Water of Arabidopsis thaliana with Mutated Atclb Gene

An Arabidopsis thaliana Ca²⁺-dependent lipid-binding protein (AtCLB) containing C2 domain is able to negatively regulate stress response in Arabidopsis thaliana. Expression of the Atclb gene was documented in all analyzed tissues of Arabidopsis thaliana (leaf, root, stem, flower, and silique) by real-time PCR analysis. Immunofluorescence analysis revealed that AtCLB protein is localized in the nucleus of cells in Arabidopsis thaliana root tips. It is demonstrated that the AtCLB protein is capable of binding to the membrane lipid ceramide. The role of the Atclb gene in negatively regulating responses to abiotic stress in Arabidopsis thaliana was identified. FIGS. 3A-3E illustrate drought-tolerance phenotype of T-DNA insertion knockout lines Atclb1-1 and Atclb1-2 produced according to one embodiment of the present invention.

Two T-DNA insertion knockout mutant lines Atclb1-1 and Atclb1-2 were constructed. The mutant lines Atclb1-1 and Atclb1-2 were confirmed homozygous mutant lines. FIG. 3A shows schematically the AtCLB genomic structure, with T-DNA insertion site indicated for Atclb-1. Referring to FIG. 3A, 5′UTR and 3′UTR are 5′ untranslated region and 3′ untranslated region respectively. The start codon ATG and stop codon TGA are labeled. The box and solid lines indicate exons and introns, respectively, and E1-E11 represent 11 exons. The insertion position of the T-DNA in the Atclb1-1 mutant line is indicated by a triangle, which pointed to exon 10.

FIG. 3E shows expression analysis of wild type and two homozygous knockout lines Atlcb1-1 and Atclb1-2 of Arabidopsis thaliana by reverse transcription polymerase chain reaction (RT-PCR). Actin was used as the internal control. As shown in FIG. 3E, transcript of the Atclb gene was detected in the wild type (WT) Arabidopsis thaliana, while no transcript of the Atclb gene was detected in either of the knockout lines Atlcb1-1 and Atclb1-2.

Referring to FIG. 3B, germination rate of Arabidopsis thaliana wild type (WT), a line expressing Atclb under the control of the 35S promoter, and two knockout mutant lines Atclb1-1 and Atclb1-2 after 1 week of incubation on MS medium are shown. The mutant lines Atclb1-1 and Atclb1-2 exhibit higher germination rate (100%) compared with wild-type plants (69.2%±4%). Further, the mutant lines produced slightly longer roots.

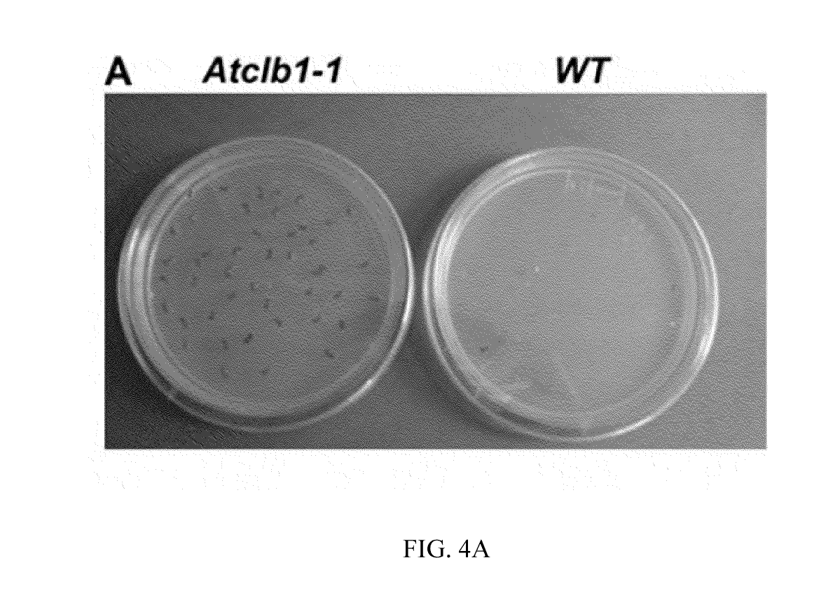

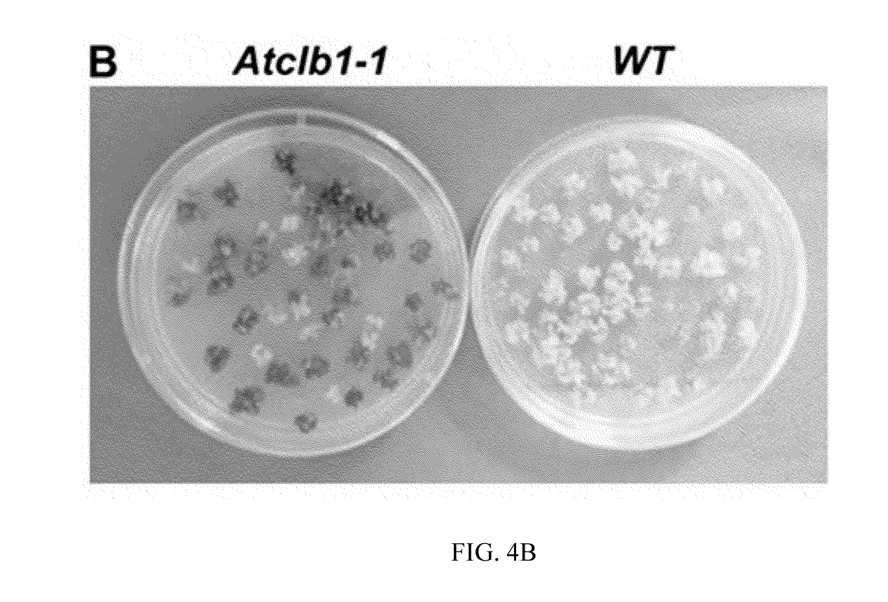

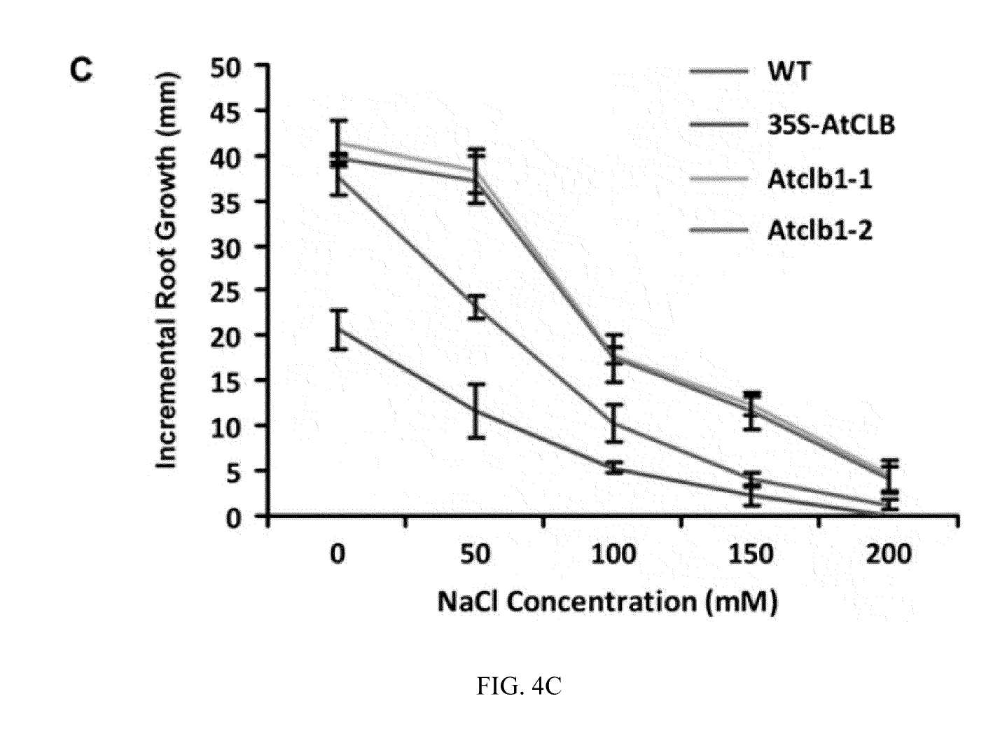

The loss of the Atclb gene function in mutants confers an enhanced drought tolerance (FIGS. 3A-3E), salt tolerance (FIGS. 4A-4C), and a modified gravitropic response (not shown) in T-DNA insertion knockout mutant lines. To test water deficit stress response, 4-week-old wild-type and mutant plants (Atclb1-1 and Atclb1-2) were deprived of water for 3 weeks in a growth chamber under a 16-hour photoperiod at 23/23° C. (day/night). At the end of the treatment period, wild-type plants showed signs of water deficit stress as indicated by wilting and yellowing of their rosette leaves, while no stress symptoms were observed in mutant plants (FIG. 3D). Furthermore, plants from both mutant lines (Atclb1-1 and Atclb1-2) were able to maintain their leaf Relative Water Content (RWC) even at 3 weeks of withholding water compared with wild-type plants which showed a rapid decrease in leaf RWC from as early as 1 week after withholding water (FIG. 3C). Hence the observed phenotypic differences between wild-type and mutant plants in relation to water deficit stress tolerance may be attributed to the differences in expression of the Atclb gene (FIG. 3E).

### Example 2

### Tolerance to Salt of Arabidopsis thaliana with Mutated Atclb Gene

Referring to FIGS. 4A-4C, tolerance of mutant lines Atclb1-1 and Atclb1-2 to salt are evaluated. 1-week-old seedlings of wild type, 35S-Atclb overexpression line, Atclb1-1, and Atclb1-2 mutant lines were grown on media containing different concentrations of NaCl. The Atclb knockout lines exhibited increased tolerance to salt stress, while 35S-Atclb overexpression lines were hypersensitive to NaCl. Referring to FIG. 4A, seeds of the Atclb1-1 knockout line were able to germinate on 150 mM NaCl-containing medium. Further, referring to FIG. 4B, sterilized seeds of wild type and knockout lines were germinated on regular MS medium, then 6-day-old seedlings were transferred to Petri plates containing 150 mM NaCl. The seedlings of loss-of-function Atclb1-1 mutant line were able to survive in the presence of 150 mM NaCl for 2 months.

Referring to FIG. 4C, in a separate experiment, one-week-old Arabidopsis thaliana seedlings with 10 to 20 mm long roots on vertical agar plates were transferred to plates supplemented with NaCl and allowed to grow vertically at 23° C. with a 16-hour light period. After 1 week of stress, incremental root growth of each seedling was recorded. Although the incremental root growth of all plants decreased with the increasing salt concentration in the media, both mutant lines exhibited higher root growth compared with wild-type and 35S-Atclb overexpression lines. The incremental root growth of 35S-Atclb plants was less than that of wild-type seedlings and reduced to almost zero at 200 mM NaCl concentration.

According to Examples 1 and 2, it is demonstrated that loss of function of the Atclb gene can lead to increased tolerance to abiotic stress (salt and drought) in Arabidopsis thaliana plant.

### Example 3

### Constructing a Transgenic Rice Plant Tolerant to Abiotic Stress by Silencing OsHP Gene

Atclb gene is playing role of negative regulator of abiotic stress response. In certain embodiments, the silencing of active homologues of Atclb gene will lead to increase of tolerance to abiotic stress. The plant or crop of choice includes, but not limited to, Solanum lycopersicum, Ricinus communis, Populus trichocarpa, Vitis vinifera, Oryza sativa, and Sorghum bicolor.

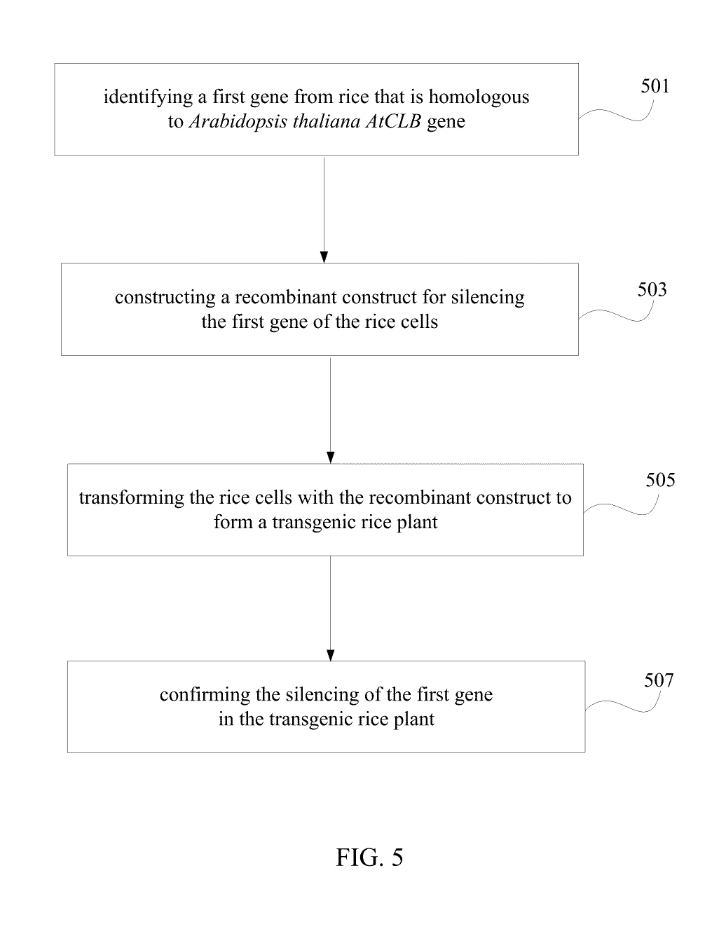

FIG. 5 illustrates a method for producing a rice plant tolerant to stress conditions by silencing a gene that contains C2 domain that is homologue to C2 domain of Atclb gene, according to one embodiment of the present invention. Vectors for post transcriptional gene silencing (RNA interference (RNAi)) or virus-induced gene silencing (VIGS) can be used for silencing of AtCLB crop homologues.

In the embodiment, Rice cultivar Nipponbare or other rice cultivar is used as the starting material for constructing a transgenic rice plant.

In step 501, through bioinformatics analysis, a first gene, rice OsHP gene, is identified as homologous to Atclb gene of Arabidopsis thaliana based on homology of C2 domains.

In step 503, a recombinant construct is established. In one embodiment, a fragment of C2 domain (300 base pair) of OsHP gene was cloned into special silencing pANDA vector (RNAi vector) under control strong promoter (uniquitin-1 (ubi)) to form the recombinant construct.

In step 505, rice plant cells or rice plants are transformed with the established recombinant construct to form transformed rice cells, and produce a transgenic rice plant from the transformed cells.

In step 507, the silencing of the first gene in the transgenic rice plant is confirmed by analysis of expression of OsHP gene.

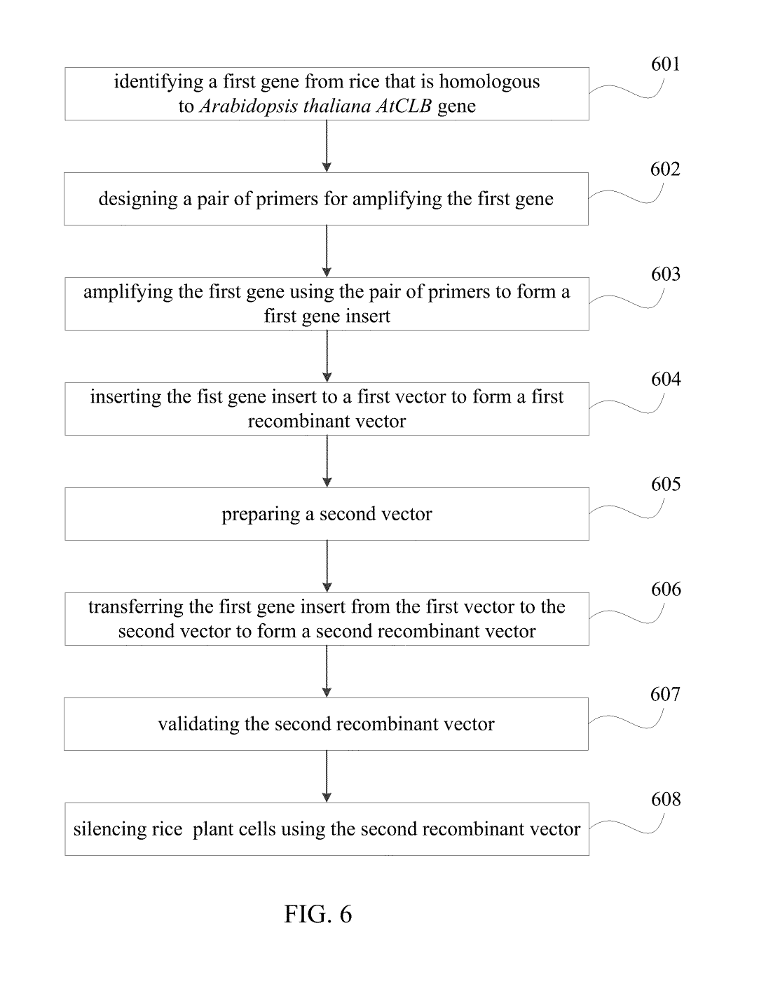

A specific example is shown in FIG. 6. FIG. 6 illustrates a method for producing a rice plant tolerant to stress conditions by silencing a gene that contains C2 domain that is homologue to C2 domain of Atclb gene, according to one embodiment of the present invention. Vectors for post transcriptional gene silencing (RNA interference (RNAi)) or virus-induced gene silencing (VIGS) can be used for silencing of AtCLB crop homologues.

In step 601, through bioinformatics analysis, a rice OsHP gene, was identified as homologous to Atclb gene of Arabidopsis thaliana based on homology of C2 domains. Sequence information for the OsHP gene was obtained from the National Center for Biotechnology Information (Tanaka et al., 2010) Nucleotide Database under the accession number NM_001065974, which is a complete coding sequence (CDS) of Oryza sativa Japonica Group Os07g0409100 (Os07g0409100). The 276 base pair (bp) OsHP gene fragment was identified from position 1445 to 1720 of the above CDS, and is listed as SEQ ID NO:10. The 276 bp OsHP gene or 276 bp OsHP gene fragment includes 32 bases downstream of the stop codon.

In step 602, a pair of primers were designed for amplifying the 276 bp OsHP gene fragment. Possible regions for forward primer sequences were verified for alignment specificity using the Basic Local Alignment Search Tool (BLAST) in the Gramene database (BLAST Search). Pairwise primer sequences were designed using an online primer designing tool (Primer-BLAST). Primer sequences were analyzed with Integrated DNA Technologies Oligo Analyzer Tool (Oligo Analyzer 3.1). The sequences of the final primer pair selected are:

(forward primer, SEQ ID NO: 12)
5′-CACCGTTGGACTTGTGGGCACT-3′,
and
(reverse primer, SEQ ID NO: 13)
5′-TGCGATGTCCATTGCAATCACTGTA-3′.

The primers listed above were used to amplify the cloning insert, the 276 bp OsHP gene fragment. The original first four bases of TGGT on the 5′ end of the forward primer were replaced with CACC to facilitate directional Gateway® Cloning.

In step 603, a 276 bp OsHP gene polymerase chain reaction (PCR) product were obtained by PCR using the designed forward primer and the reverse primer. Specifically, the step of 503 includes isolating total RNA, synthesizing cDNA from the isolated total RNA, and then using the obtained cDNA as template and the designed pair of primers to obtain sufficient amount of OsHP gene PCR product through PCR.

Firstly, frozen leaf tissue of LaGrue rice (Oryza sativa L.) was used for isolating the total RNA using the RNeasy® Plant Mini Kit (Qiagen Inc., Germantown, Md.).

Then, the isolated total RNA was used to synthesize complementary DNA (cDNA) according to kit protocol using the SuperScript® III First Strand Synthesis System Kit (Invitrogen) with oligo(dT) primers.

Following cDNA synthesis, PCR was performed to amplify the OsHP gene fragment to obtain the 276 bp OsHP gene PCR product or the OsHP insert, where the cDNA of LaGrue rice obtained from the above step was used as the PCR template, and the SEQ ID:12 and SEQ ID NO:13 were used as the forward and reverse primer. The PCR was performed using high fidelity Platinum® Pfx DNA Polymerase (Invitrogen) in 35 cycles of a traditional 3-step PCR protocol with an annealing temperature of 59° C. and extension temperature of 68° C. The PCR product was resolved on a 1% agarose gel in 1×Tris/Borate/EDTA (TBE), stained with ethidium bromide. The gel was visualized and recorded using a Gel Doc™ XR Gel Imaging System (Bio-Rad Life Science Research, Hercules, Calif.). The PCT product might be purified from gele extraction, or other methods such as PEG precipitation, ethanol precipitation, or column purification. In certain embodiments, the PCR product might be used directly without further purification. The obtained OsHP gene PCR product, or the OsHP insert, contains the 276 bp OsHP gene fragment with the first four nucleotides changed, which was used to construct the entry vector for Gateway® cloning. The OsHP gene PCR product or the OsHP insert has the sequence of SEQ ID NO:11.

In step 604, the entry clone containing the OsHP insert was constructed using the OsHP insert obtained through PCR (SEQ ID NO:11) and entry vector pENTR™/D-TOPO®.

The entry clone was prepared using a Gateway® cloning kit (Invitrogen) containing the entry vector pENTR™/D-TOPO®, for example, pENTR™/D-TOPO®, Cloning Kit with One Shot® TOP10 Chemically Competent E. coli. The 276 bp PCR product of OsHP insert (SEQ ID NO:11) was used as the insert DNA in the cloning reaction performed according to kit instructions. Specifically, according to the kit protocol, the OsHP insert, the pENTR™/D-TOPO® vector, a control, and buffers were mixed to form a first sample, and BP Clonase™ enzyme was added to the first sample to form the reaction mixture. The reaction mixture was incubated for a certain period of time, for example 1 hour, and then the reaction was stopped by adding Proteinase K solution and incubated for a certain period of time, for example 10 minutes. The reaction mixture was then used to transform One Shot® TOP10 Chemically Competent E. coli (Invitrogen) using a standard heat-shock protocol at 42° C. The transformed competent cell was then cultured on petri plates (100×15 mm) of Luria Broth (LB) agar medium containing kanamycin (50 mg/L) for selection of transformed colonies. All colonies grow on the petri plates were confirmed to have the OsHP insert containing the SEQ ID NO:11 through PCR analysis using the insert specific primer pair (that is, the primers of SEQ ID NO:12/SEQ ID NO:13). Single colonies with the most robust PCR amplification were used to inoculate selective (50 mg/L kanamycin) LB liquid cultures for bacterial growth and subsequent isolation of purified pENTR™/D-TOPO®_OsHP entry clone. Cultures were incubated shaking at room temperature. The bacterial cells were pelleted, and the plasmid was isolated using a QIAfilter™ Plasmid Midi kit (Qiagen, Inc.) according to kit instructions. Purified plasmid stocks were analyzed by PCR using the OsHP specific primers and with a BcgI/NotI double restriction digest to confirm the presence of the OsHP insert and correct digestion band pattern. Though E. coli is normally cultured at 37° C., it is critical that the present invention uses room temperature culturing, so that the plasmid production is stable from the cells.

In step 605, a destination vector was prepared. The pANDA silencing Gateway® destination vector (Shimamoto, 2004) was used to transform One Shot® ccdB Survival™ 2 T1^{R} Competent E. coli (Invitrogen) by a standard heat shock protocol at 42° C. The transformed One Shot ccdB Survival™ 2T1R Competent E. coli was grown on kanamycin and hygromycin B (50 mg/L each antibiotic) selective LB agar plates. Single colonies grown on the LB agar plate were used to inoculate 100 ml selective LB liquid cultures for plasmid growth. Plasmid were isolated from the LB liquid culture using the QIAfilter™ Plasmid Midi kit (Qiagen, Inc.) according to kit instructions. Purified pANDA plasmid stocks were analyzed by PCR with the insert specific OsHP primers to confirm that the insert did not amplify, or in other words, the OsHP is not present in the destination vector. Double restriction digestion was performed using KpnI and SacI restriction endonucleases to confirm the correct digestion pattern. Digestion and PCR products as described above were resolved on a 1% agarose gel in 1× Tris/Borate/EDTA (TBE), and stained with ethidium bromide. The stained gel was visualized and recorded using a Gel Doc™ XR Gel Imaging System (Bio-Rad).

In step 606, the OsHP insert contained in the entry vector was transferred to the destination vector through LR reaction, and the produced construct that contains the OsHP insert was used to transform E. coli.

Invitrogen's Gateway® LR Clonase™ Enzyme Mix was used to transfer the OsHP insert from the pENTR™/D-TOPO entry clone prepared in step 604 to the pANDA destination vector prepared in step 605. Specifically, the pENTR™/D-TOPO entry clone, the pANDA destination vector, positive control vector, were mixed as a second sample. LR Clonase™ enzyme mix was added to the second sample to form a reaction mixture. The reaction mixture was incubated for an overnight incubation at room temperature, the reaction was stopped with the addition of Proteinase K, and then the produced construct was used to transform One Shot® TOP10 Competent E. coli using the standard 42° C. heat shock protocol. The produced construct was named pANDA OsHP DNA construct. The transformed cells were divided into three aliquots of 50 μl, 100 μl, and 150 μl and spread onto three separate selective LB agar plates containing 50 mg/L of each of the antibiotics kanamycin and hygromycin B.

In step 607, the transformed E. coli was tested. Resistant isolated colonies were tested for the presence of the OsHP insert using PCR with the OsHP insert specific primers. Confirmed colonies were selected for growth in 100 ml LB medium (kanamycin/hygromycin selective) and subsequent plasmid isolation. Liquid cultures were incubated at room temperature with shaking. Plasmid purifications were accomplished using the QIAfilter™ Plasmid Midi kit (Qiagen, Inc.) according to kit instructions, and the purified pANDA OsHP DNA stocks were confirmed to have the OsHP insert through PCR analysis and double restriction digestion using KpnI and SacI restriction endonucleases. Digestion product and PCR product were resolved on a 1% agarose gel in 1×TBE, and stained with ethidium bromide. The gel was visualized and recorded using a Gel Doc™ XR Gel Imaging System (Bio-Rad). Though E. coli is normally cultured at 37° C., it is critical that the present invention uses room temperature culturing, so that the plasmid production is stable from the cells.

In step 608, silencing of rice OsHP gene using the above obtained pANDA OsHP DNA construct. The pANDA OsHP DNA construct was used through Agrobacterium mediated transformation to transform rice plants. Silencing of the OsHP gene of the rice plant was confirmed, and the transformed rice plant shown resistance to abiotic stress, including water deficit, salt stress, cold stress or a combination thereof.

It is important for the silencing pANDA OsHP DNA construct to have the 276 bp OsHP gene fragment as shown in SEQ ID NO: 11, which showed efficient silencing when comparing with other designs that have varies regions or lengths of the OsHP gene.

In one embodiment, a seed was produced from the transformed rice plant.

In certain embodiments, the recombinant construct was capable of silencing the rice OsHP gene, and the transformed or transgenic rice plant shown increased abiotic stress tolerance comparing to wild type rice plant.

In this embodiment, firstly, it is critical to identify the 276 bp nucleotide sequence having SEQ ID NO:10, amplify the nucleotide sequence, insert the amplified nucleotide sequence to the entry vector, and transfer it to the destination vector, where the vectors contains the insert having 276 bp nucleotide sequence of SEQ ID NO:11. It is found that by introducing the above constructed vector containing the nucleotide sequence having SEQ ID NO:11 to Oryza sativa, efficient silencing of OsHP gene of the Oryza sativa plant or plat cells were observed. As a result, the Oryza sativa plant or plat cells shows resistance to abiotic stress due to the silencing of the OsHP gene.

Secondly, when the PCR product was inserted into the entry vector of pENTR™/D-TOPO®, and the recombinant vector was used to transform E. coli cells, it is critical to incubate the E. coli cell culture at room temperature instead of 37° C. The incubation of the E. coli cell culture at room temperature ensures stable amplification of the recombinant entry vector. In contrast, if the E. coli cell culture was incubated at 37° C., the cells are prone to lost their recombinant entry vectors during the incubation process.

Thirdly, when the OsHP insert was transferred from the entry vector to the destination vector, and the recombinant destination vector was used to transform E. coli cells, it is critical to incubate the E. coli cell culture at room temperature instead of 37° C. The incubation of the E. coli cell culture at room temperature ensures stable amplification of the recombinant destination vector. In contrast, if the E. coli cell culture was incubated at 37° C., the cells are prone to lost their recombinant entry vectors during the incubation process.

### Example 4

### Constructing a Transgenic Tomato Plant Tolerant to Abiotic Stress by Silencing SlCLB1 Gene

In one embodiment, the plant of choice is tomato plant Solanum lycopersicum.

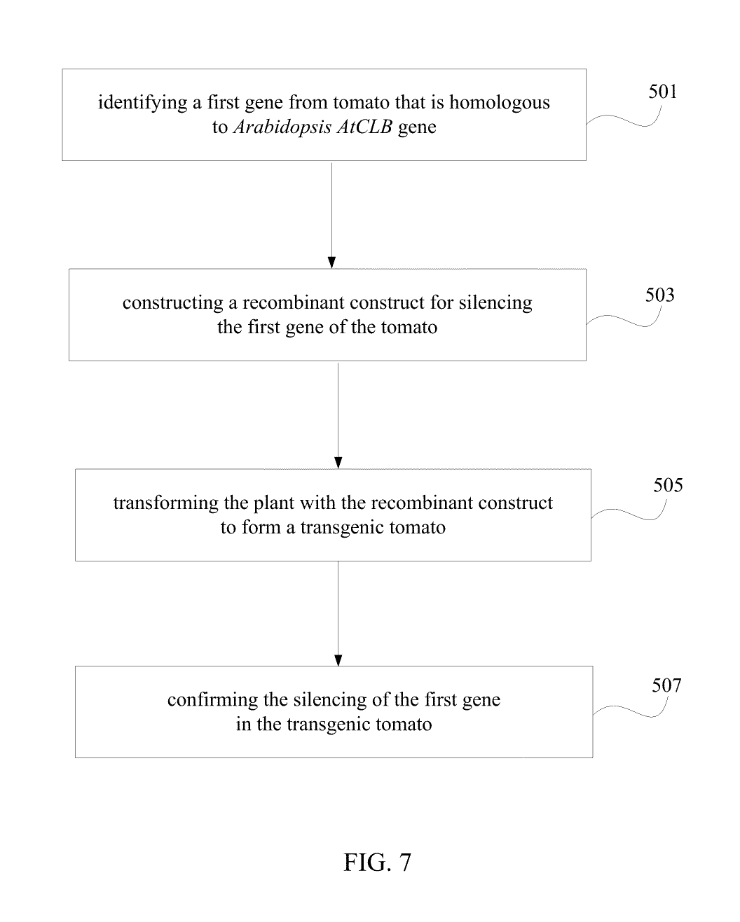

FIG. 7 illustrates a method for producing a tomato plant tolerant to abiotic stress conditions by silencing a gene homologue that is homologous to Arabidopsis thaliana Atclb gene, according to one embodiment of the present invention.

In step 701, through bioinformatics analysis, a first gene, rice SlCLB1 gene, is identified as homologous to Atclb gene of Arabidopsis thaliana based on homology of C2 domains.

In step 703, a recombinant construct is established. In one embodiment, a fragment of SlCLB1 gene, corresponding to a polynucleotide of Atclb gene encoding at least part of C2 domain, is cloned into any suitable silencing vector to form the recombinant construct.

In step 705, tomato plant cells are transformed with the established recombinant construct to form transformed tomato cells, and produce a transgenic tomato plant from the transformed cells.

In step 707, the silencing of the first gene in the transgenic tomato plant is confirmed.

In one embodiment, a seed is produced from the transgenic tomato plant.

In certain embodiments, the recombinant construct is capable of silencing the tomato SlCLB1 gene, and the transgenic tomato plant shows increased stress tolerance comparing to wild type tomato plant.

The foregoing description of the exemplary embodiments of the invention has been presented only for the purposes of illustration and description and is not intended to be exhaustive or to limit the invention to the precise forms disclosed. Many modifications and variations are possible in light of the above teaching.

While there has been shown several and alternate embodiments of the present invention, it is to be understood that certain changes can be made as would be known to one skilled in the art without departing from the underlying scope of the invention as is discussed and set forth above and below including claims and drawings. Furthermore, the embodiments described above and claims set forth below are only intended to illustrate the principles of the present invention and are not intended to limit the scope of the invention to the disclosed elements. Moreover, aspects of the present invention are further disclosed and described in Appendix A, which are incorporated herein by references in their entireties as an integral part of the application.

What is claimed is:

1. A method comprising:
identifying a first nucleotide sequence from Oryza sativa, wherein the first nucleotide sequence is homologous to a Ca²⁺-dependent lipid-binding gene of Arabidopsis thaliana (Atclb gene);
cloning the first nucleotide sequence to a first vector to form a first recombinant vector, such that the first recombinant vector contains a first insert of the first nucleotide sequence; and
transfer the first insert in the first recombinant vector to a second vector to form a second recombinant vector, such that the second recombinant vector contains a second insert of the first nucleotide sequence,
wherein the second recombinant vector, when being introduced to Oryza sativa plant cells, is capable of silencing the first nucleotide sequence of the Oryza sativa plant cells, such that the Oryza sativa plant cells are tolerant to an abiotic stress.

2. The method of claim 1, wherein the first nucleotide sequence is homologous to nucleotide sequence 963-1205 of SEQ ID NO:1.

3. The method of claim 1, wherein the first nucleotide sequence encodes Oryza sativa histidine-containing phosphotransfer protein, and has the sequence of SEQ ID NO: 10.

4. The method of claim 1, wherein the first vector is an entry vector pENTR™/D-TOPO, and the step of cloning the first nucleotide sequence to the first vector is a BP reaction.

5. The method of claim 1, wherein the first insert has the sequence of SEQ ID NO: 11.

6. The method of claim 1, further comprising transforming E. coli competent cells by the first recombinant vector to form transformed E. coli competent cells, wherein the transformed E. coli competent cells were incubated at room temperature.

7. The method of claim 1, further comprising transforming E. coli competent cells by the second recombinant vector to form transformed E. coli competent cells, wherein the transformed E. coli competent cells were incubated at room temperature.

8. The method of claim 1, wherein the second vector is a pANDA destination vector, and the step of transfer the first insert in the first recombinant vector to the second vector is an LR reaction.

9. The method of claim 1, further comprising transforming the Oryza sativa plant cells using the second recombinant vector to form transformed Oryza sativa plant cells.

10. The method of claim 9, wherein the Oryza sativa plant cells are transformed by agrobacterial transformation or bombardment transformation to form the transformed Oryza sativa plant cells.

11. The method of claim 1, further comprising:
before the step of cloning the first nucleotide sequence to the first vector to form the first recombinant vector, amplifying the first nucleotide sequence using a pair of primers that have the nucleotide sequences of SEQ ID NO:12 and SEQ ID NO:13.

12. The method of claim 1, wherein the abiotic stress is water deficit, salt stress, cold stress or a combination thereof.

13. The method of claim 1, wherein the first insert and the second insert have the same nucleotide sequence.


SEQUENCE LISTING (13 sequences)

SEQ ID NO: 1 - DNA, 1806 bp, Arabidopsis thaliana
aatgcgttcttaattttgttctgattctccccacttgaccaaccccgtgaggataaactgttgccgacgcttctcgccgataacaccggttttgttgtgacggaaaggaaaaatgggtttgatttctgggattctgttcgggattatcttcggcgtagctctaatggctggctggagtcgtatgatgactcaccgatcgtccaagcgagtcgccaaggcagttgatatgaaactactaggctctcttagcagagacgatttgaagaaaatttgtggtgataatttccctcaatggatatcgtttccagcatttgaacaggtcaaatggctgaataaactactgagcaagatgtggccatatattgcagaggcagcaacaatggtaataagagattctgttgaacctctgcttgaagattacagaccaccaggaattacttccctgaagttcagcaaattgacgttgggtaacgtagcaccaaagattgaaggtattcgtgttcaaagtttcaaggaaggtcaagtcacaatggacgttgatcttcgatggggtggtgatccaaatattgttttaggtgtgacagcacttgttgcctccatacccattcagttgaaggatcttcaagttttcacagttgcacgtgttatctttcaacttgcggatgagattccttgtatatctgctgttgttgttgctctacttgctgagccaaaaccaagaattgattacactctgaaggctgttggtggaagcttaacagcaattcctggactatctgatatgattgatgatactgttgatactatcgtcaaagacatgcttcagtggcctcatagaattgttgttcccattggtggtattcctgttgatttaagtgatttggagcttaagccacagggaaagcttatagtaacagttgtgaaagcaactaacttgaagaataaggaattgattggaaaatctgacccttatgctaccatctacattcgtcctgtattcaagtataaaacaaaggcaatcgagaacaatctgaatcctgtctgggatcaaacattcgaattgattgcagaggacaaagaaacccagtcgctcactgtagaggtatttgataaagacgtaggtcaagatgagcgccttggacttgtgaaacttcccttaagcagtttggaagccggagttacaaaagaactggagctaaatctgttgtcttcacttgatactttgaaagtaaaagataagaaagatagaggaagcataactcttaaggtacattatcatgagttcaacaaagaggagcaaatggctgcgttggaagacgagaagaagatcatggaagaaaggaagagactgaaggaagcaggagtgataggtagcacaatggatgcagtcgggatggtgggaagtgggctcggtgctggtgtaggaatggttgggaccggtattggcacaggagtcggattggttggaagcggtgtgagctcgggtgttgggatggttggtagcggttttggagcggtcggtagtggattgagcaaagcagggagatttatgggaagaacaatcacaggtcagtctagcaaacgtagtggctcctcaacacctgtgaataccgttccagaaaacgatggtgcaaaacagcagtgagtaaactttgggtttaagcaagatttgtgatcatgacttcgtttttactctttattgttctgtttttttcccctttaactctctcgaacttgatttctggattcactgcagtaatttgttttcgttgtgagccttcaaattaaaatcttgtacaaaaaaaa

SEQ ID NO: 2 - protein, 510 aa, Arabidopsis thaliana
MGLISGILFGIIFGVALMAGWSRMMTHRSSKRVAKAVDMKLLGSLSRDDLKKICGDNFPQWISFPAFEQVKWLNKLLSKMWPYIAEAATMVIRDSVEPLLEDYRPPGITSLKFSKLTLGNVAPKIEGIRVQSFKEGQVTMDVDLRWGGDPNIVLGVTALVASIPIQLKDLQVFTVARVIFQLADEIPCISAVVVALLAEPKPRIDYTLKAVGGSLTAIPGLSDMIDDTVDTIVKDMLQWPHRIVVPIGGIPVDLSDLELKPQGKLIVTVVKATNLKNKELIGKSDPYATIYIRPVFKYKTKAIENNLNPVWDQTFELIAEDKETQSLTVEVFDKDVGQDERLGLVKLPLSSLEAGVTKELELNLLSSLDTLKVKDKKDRGSITLKVHYHEFNKEEQMAALEDEKKIMEERKRLKEAGVIGSTMDAVGMVGSGLGAGVGMVGTGIGTGVGLVGSGVSSGVGMVGSGFGAVGSGLSKAGRFMGRTITGQSSKRSGSSTPVNTVPENDGAKQQ

SEQ ID NO: 3 - protein, 81 aa, Arabidopsis thaliana
LIVTVVKATNLKNKELIGKSDPYATIYIRPVFKYKTKAIENNLNPVWDQTFELIAEDKETQSLTVEVFDKDVGQDERLGLV

SEQ ID NO: 4 - protein, 82 aa, Solanum lycopersicum
LTVTIVKANGLKNHEMIGKSDPYAVVHIRPLFKVKTKTIDNNLNPVWDQTFELIAEDKETQSLFIEVFDKDNIGQDQRMGVA

SEQ ID NO: 5 - protein, 80 aa, Ricinus communis
LTVTVVKANDLKNMEMIGKSDPYVVLYIRPMFKVKSRTIENNLNPVWDQTFEMIAEDKETQSLILEVFDKDIGQDKRLGI

SEQ ID NO: 6 - protein, 82 aa, Populus trichocarpa
LTVTVVKANDLKNMEMIGKSDPYAVVYIRPMFKVKTQVVDNNLNPVWNQTFDLIAEDKETQSLILEVFDKDIGQDKRLGRAK

SEQ ID NO: 7 - protein, 80 aa, Vitis vinifera
LTLTIVKANDLKNMEMIGKSDPYVVVHIRPLFKIKTKVIENNLNPVWNQTFELIAEDKETQSLILEVIDKDITQDKRLGI

SEQ ID NO: 8 - protein, 82 aa, Oryza sativa
LTVTVVKATSLKNKELIGKSDPYVILYVRPMFKVKTKVIDDNLNPEWNETFPLIVEDKETQSVIFEVYDEDRLQQDKKLGVA

SEQ ID NO: 9 - protein, 82 aa, Sorghum bicolor
LAVTVVKATCLVNMEMIGKSDPYVVLYIRPMLKVKTKVVDHNLNPEWNETFHLIVEDKETQEVIFEIYDEDTLQQDKKMGVA

SEQ ID NO: 10 - DNA, 276 bp, Oryza sativa
tggtgttggacttgtgggcactggcattgtcggcggggttggacttgttggatcaggaattggtgctggtgttgggcttgttggttcgggtgttgggcttgttggttcgggtattggcgctgtcggcagcggcctcggtaaagctgggaaattcatgggcaagactgtggccgggcctttcagtatgtcccggaagaacggtagcagctcaactgctccccaggctgaacaaccttctgcgtgacttgatgtacagtgattgcaatggacatcgca

SEQ ID NO: 11 - DNA, 276 bp, Oryza sativa
caccgttggacttgtgggcactggcattgtcggcggggttggacttgttggatcaggaattggtgctggtgttgggcttgttggttcgggtgttgggcttgttggttcgggtattggcgctgtcggcagcggcctcggtaaagctgggaaattcatgggcaagactgtggccgggcctttcagtatgtcccggaagaacggtagcagctcaactgctccccaggctgaacaaccttctgcgtgacttgatgtacagtgattgcaatggacatcgca

SEQ ID NO: 12 - DNA, 22 bp, Artificial Sequence
  Forward Primer for OsHP gene
caccgttggacttgtgggcact

SEQ ID NO: 13 - DNA, 25 bp, Artificial Sequence
  Reverse primer for OsHP gene
tgcgatgtccattgcaatcactgta